(19) United States
(12) Patent Application Publication
(10) Pub. No.: US 2012/0071464 A1
(43) Pub. Date: Mar. 22, 2012

(54) METHOD OF TREATMENT OF OBSESSIVE COMPULSIVE DISORDER WITH ONDANSETRON

(76) Inventor: NIKHILESH N. SINGH, Mill Valley, CA (US)

(21) Appl. No.: 13/244,811

(22) Filed: Sep. 26, 2011

Related U.S. Application Data
(63) Continuation of application No. 12/782,571, filed on May 18, 2010.
(60) Provisional application No. 61/179,439, filed on May 19, 2009.

(51) Int. Cl. A61K 31/55; A61K 31/4178; A61P 25/00; A61K 31/4525

(52) U.S. Cl. 514/217; 514/321; 514/397

(57) ABSTRACT

Methods for treating obsessive compulsive disorder are described. In one method, a serotonin reuptake inhibitor (SRI) and ondansetron or a pharmaceutically acceptable salt thereof is administered to a patient suffering from obsessive compulsive disorder. The step of administering the SRI and the ondansetron is then repeated for more than seven days. In another method, an SRI, a neuroleptic, and ondansetron or a pharmaceutically acceptable salt thereof is administered to a patient suffering from obsessive compulsive disorder. The step of administering the SRI, the neuroleptic, and the ondansetron is then repeated for more than seven days. In another method, ondansetron or a pharmaceutically acceptable salt thereof is administered to a patient suffering from obsessive compulsive disorder for more than seven days. The ondansetron or pharmaceutically acceptable salt thereof may be administered as a pharmaceutically effective dose up to about 1.5 mg (free-base equivalent).

### CROSS-REFERENCES TO RELATED APPLICATIONS

This application is a continuation of U.S. application Ser. No. 12/782,571, which claims priority to U.S. Provisional Application Ser. No. 61/179,439, filed May 19, 2009, entitled “Method of Treatment of Obsessive Compulsive Disorder with Ondansetron,” both of which are hereby expressly incorporated by reference in their entirety.

### BACKGROUND OF THE INVENTION

Obsessive compulsive disorder (“OCD”) is a mental condition that is most commonly characterized by intrusive, repetitive unwanted thoughts (obsessions) resulting in compulsive behaviors and mental acts that an individual feels driven to perform (compulsion). Current epidemiological data indicates that OCD is the fourth most common mental disorder in the United States. Some studies suggest the prevalence of OCD is between one and three percent, although the prevalence of clinically recognized OCD is much lower, suggesting that many individuals with the disorder may not be diagnosed. Patients with OCD are often diagnosed by a psychologist, psychiatrist, or psychoanalyst according to the DSM-1V-TR (2000) diagnostic criteria that include six characteristics of obsessions and compulsions:

Obsessions
Recurrent and persistent thoughts, impulses, or images that are experienced as intrusive and that cause marked anxiety or distress.
The thoughts, impulses, or images are not simply excessive worries about real-life problems.
The person attempts to ignore or suppress such thoughts, impulses, or images, or to neutralize them with some other thought or action.
The person recognizes that the obsessional thoughts, impulses, or images are a product of his or her own mind, and are not based in reality.

Compulsions
Repetitive behaviors or mental acts that the person feels driven to perform in response to an obsession, or according to rules that must be applied rigidly.
The behaviors or mental acts are aimed at preventing or reducing distress or preventing some dreaded event or situation; however, these behaviors or mental acts are not actually connected to the issue, or they are excessive.

Individuals with OCD typically perform tasks (or compulsion) to seek relief from obsession-related anxiety. Repetitive behaviors such as handwashing, counting, checking, or cleaning are often performed with the hope of preventing obsessive thoughts or making them go away. Performing these “rituals,” however, only provides temporary relief. People with OCD may also be diagnosed with a spectrum of other mental disorders, such as generalized anxiety disorder, anorexia nervosa, panic attack, or schizophrenia.

Studies suggest that OCD may be related to abnormal levels of a neurotransmitter called serotonin. The first-line treatment of OCD consists of behavioral therapy, cognitive therapy, and medications. Medications for treatment include serotonin reuptake inhibitors (SRIs) such as paroxetine (Seroxat™, Paxil®, Xetanor™, ParoMerck™, Rexetin™), sertraline (Zoloft®, Stimuloton™), fluoxetine (Prozac®, Bioxetin™), escitalopram (Lexapro®), and fluvoxamine (Luvox®) as well as the tricyclic antidepressants, in particular clomipramine (Anafranil®). Benzodiazepines are also used in treatment. As much as 40 to 60% of the patients, however, fail to adequately respond to the SRI therapy and an even greater proportion of patients fail to experience complete remission of their symptoms.

As a second-line treatment for those patients (50-60%) who do not respond to first-line treatment (SRIs), atypical antipsychotics (or neuroleptics) may be used as augmentation in treatment-resistant OCD. These atypical antipsychotics block central dopamine receptors and may be helpful in blocking mid-brain dopamine receptors. Low doses of atypical antipsychotics, such as olanzapine (Zyprexa®), quetiapine (Seroquel®), ziprasidone, and risperidone (Risperdal®), have been found useful as adjuvant therapy with SRIs in the treatment of OCD. Other alternative agents, including inositol, vitamin supplement, opioids, hallucinogen LSD, and nicotine, have been attempted as a drug treatment for OCD. The efficacy of the adjunctive antipsychotics with SRIs may be due to direct D₂ blockade separate or together with antagonism of 5-HT₂ receptors. These patients may also suffer from additional abnormalities in dopaminergic function that require augmentation with these dopamine-blocking agents. Still, only a third to half of patients with SRI-resistant (treatment-resistant) OCD had a beneficial response to this second-line treatment.

Obsessive-compulsive disorder treatment may, however, be difficult. In fact, 30% of patients do not respond to adjuvant therapy (neuroleptics plus SRIs). For these patients, current therapies do not offer a cure.

We have unexpectedly discovered alternative therapies, which include the administration of low doses of ondansetron for more than one week, that are effective for treating OCD and treatment-resistant OCD.

### SUMMARY OF THE INVENTION

The present invention provides methods and compositions for treating OCD, such as refractory patients with treatment-resistant OCD, with particularly low doses of ondansetron for multiple weeks (e.g., for one week and then on a continuing basis until beneficial effects are seen, and preferably for so long thereafter as such effects persist). Ondansetron may be used either in its free base forms or suitable salt form. The most commonly used salt form of ondansetron is ondansetron hydrochloride. The ondansetron (or pharmaceutically acceptable salt thereof) may be administered alone, with an SRI, or with an SRI and neuroleptic. The present invention may be used for, but is not limited to, OCD patients who have not responded to SRI therapy or SRI plus neuroleptic therapy. The present invention may also be used on patients who have not yet tried SRI therapy or SRI plus neuroleptic therapy.

In one embodiment, the invention provides a method of treating obsessive compulsive disorder. The method includes administering ondansetron or a pharmaceutically acceptable salt thereof to a patient suffering from obsessive compulsive disorder. The ondansetron or pharmaceutically acceptable salt thereof is administered as a pharmaceutically effective dose up to about 1.5 mg per day (free-base equivalent or base equivalent), alternatively between about 0.75 mg and about 1.5 mg per day, alternatively between about 0.8 mg and about 1.2 mg per day, alternatively about 1.0 mg per day. The step of administering the ondansetron or a pharmaceutically acceptable salt thereof is then repeated for more than seven days.

In another embodiment, the invention provides a method of treating obsessive compulsive disorder. The method includes administering a serotonin reuptake inhibitor and ondansetron or a pharmaceutically acceptable salt thereof to a patient suffering from obsessive compulsive disorder. The ondansetron or pharmaceutically acceptable salt thereof is administered as a pharmaceutically effective dose up to about 1.5 mg per day (free-base equivalent or base equivalent), alternatively about 0.25 mg/day to about 1.5 mg/day, alternatively about 0.5 mg/day to about 1.5 mg/day, alternatively of between about 0.75 mg and about 1.5 mg per day, alternatively between about 0.8 mg and about 1.2 mg per day, alternatively about 1.0 mg per day, alternatively about 1.5 mg/day. The step of administering the serotonin reuptake inhibitor and ondansetron or a pharmaceutically acceptable salt thereof is then repeated for more than seven days.

In another embodiment, the invention provides a method of treating obsessive compulsive disorder. The method includes administering a serotonin reuptake inhibitor, a neuroleptic, and ondansetron or a pharmaceutically acceptable salt thereof to a patient suffering from obsessive compulsive disorder. The ondansetron or pharmaceutically acceptable salt thereof is administered as a pharmaceutically effective dose up to about 1.5 mg per day (free-base equivalent or base equivalent), alternatively between about 0.75 mg and about 1.5 mg per day, alternatively between about 0.8 mg and about 1.2 mg per day, alternatively about 1.0 mg per day. The step of administering the serotonin reuptake inhibitor, neuroleptic, and ondansetron or a pharmaceutically acceptable salt thereof is then repeated for more than seven days.

The patient may be a refractory patient suffering from treatment-resistant OCD, i.e., the patient may not have responded to an SRI therapy or an SRI and neuroleptic therapy. Subjects who fail to respond to at least 8 weeks of treatment with an SRI are considered refractory patients having treatment-resistant OCD. “Failure to respond” means less than 25 percent reduction in Yale-Brown Obsessive Compulsive Scale (YBOCS) scores from baseline. For patients that fail to respond, YBOCs scores may stay above 24. YBOCS, which is a 10-item clinician-rated instrument for assessing the severity of obsessive-compulsive symptoms, has been shown to correlate on a weekly basis with co-morbid symptoms of anxiety and depression. (See Goodman, W. K. et al. “The Yale-Brown Obsessive Compulsive Scale.” ARCH GEN PSYCHIATRY 43: 1012-16 (November 1989), which is hereby expressly incorporated by reference in its entirety for all purposes.) It is sensitive to drug-induced changes in OCD patients and used as an outcome measure in drug trials for OCD.

In another embodiment, the present invention also provides compositions and methods for treating treatment-resistant OCD with ondansetron at doses between about 0.75 mg and about 1.5 mg for more than one week. Ondansetron may be used either in its free base forms or a suitable salt form, e.g., ondansetron hydrochloride.

The ondansetron or pharmaceutically acceptable salt thereof may be administered for more than 1 week, alternatively at least 2 weeks, alternatively at least 8 weeks, alternatively at least 16 weeks.

The ondansetron dosage may be administered in a single daily dose, alternatively twice a day (i.e., b.i.d.), alternatively three times a day (i.e., t.i.d.), alternatively four times a day (i.e., q.i.d.), alternatively 5 times a day, alternatively 6 times a day. For example, a 0.5 mg daily dose could be administered as a dosage of 0.25 mg twice daily. Similarly, a 1.0 mg daily dose could be administered as a dosage of 0.5 mg twice daily. Similarly, a 1.5 mg daily dose could be administered as a dosage of 0.5 mg three times a day.

A pharmaceutically effective dose of ondansetron as a free-base equivalent dose (or base equivalent dose) is a dose that achieves at least a 25 percent reduction in YBOCS scores from baseline. Such a reduction may be achieved with YBOCS scores staying at or below 24, and/or a CGI-improvement of less than or equal to 2, and/or may be achieved after at least 8 weeks (e.g., 8-12 weeks) of treatment. (See Guy, W. ed. “Clinical Global Impression (CGI)” ECDEU Assessment Manual for Psychopharmacology. Rockville, Md., U.S. Department of Health, Education, and Welfare, 1976, pp 125-126, which is hereby expressly incorporated by reference in its entirety for all purposes.) A pharmaceutically effective dose of ondansetron as a free-base equivalent dose (or base equivalent dose) from within the range from about 0.75 mg to about 1.5 mg per day, preferably about 1 mg per day, and most preferably one-half of any such dose twice per day may be administered. A pharmaceutically effective dosage is a dosage to which patients respond as against failing to respond per the definition above. The ondansetron dosage may be administered twice a day (i.e., b.i.d.). For example, a daily dose of about 0.75 mg to about 1.5 mg may be administered as a dosage of about 0.375 mg to about 0.75 mg twice daily. Similarly, a daily dose of about 1.0 mg may be administered as a dosage of about 0.5 mg twice daily. When we refer to mg doses of ondansetron, it should be understood to refer to the corresponding free-base equivalent dose or base equivalent dose unless otherwise indicated. For the twice-a-day (b.i.d.) dosage regimen, the second dose of ondansetron may be administered at least about 9 hours, alternatively at least about 10.5 hours, alternatively at least about 12 hours after the first dose of ondansetron is administered. For the b.i.d. dosage regimen, the administration of the first and second dosages may be timed such that there is no or essentially no accumulation of ondansetron in the patient over a period of about 1 week, alternatively about 2 weeks, alternatively about 3 weeks, alternatively about 4 weeks, alternatively for the period of treatment with ondansetron.

Suitable salt forms of ondansetron include, but are not limited to, ondansetron hydrochloride, hydrochloride monohydrate, hydrochloride dihydrate, hydrochloride trihydrate, hydrochloride tetrahydrate, citrate, succinate, tartrate, hydrobromide, chloride, bromide, maleate, acetate, benzoate, borate, isethionate, palmetate, oleate, salicylate, besylate, mesylate, tosylate, and sulfate. The most commonly used salt form of ondansetron is ondansetron hydrochloride.

The ondansetron may be administered such that the patient has a plasma concentration of ondansetron of between about 0.3 ng/ml and about 14 ng/ml, alternatively between about 0.5 ng/ml and about 12 ng/ml, alternatively between about 0.7 ng/ml and about 10 ng/ml. The ondansetron may also be administered such that the dose-normalized Cₘₐₓ is between about 0.5 ng/mL and about 10 ng/mL, alternatively between about 0.5 ng/mL and about 8 ng/mL.

The ondansetron may be administered such that a trough concentration for ondansetron between each dose returns essentially to baseline (i.e., is about zero or substantially undetectable).

Where the method includes administering a serotonin reuptake inhibitor in conjunction with ondansetron, the serotonin reuptake inhibitor may be, but is not limited to, clomipramine, paroxetine, sertraline, fluoxetine, escitalopram, citalopram, or fluvoxamine. Clomipramine may be administered in a dosage of about 25 mg/day to about 250 mg/day. Paroxetine may be administered in a dosage of about 20 mg/day to about 80 mg/day. Sertraline may be administered in a dosage of about 25 mg/day to about 200 mg/day. Fluoxetine may be administered in a dosage of about 20 mg/day to about 60 mg/day. Escitalopram may be administered in a dosage of about 10 mg/day to about 20 mg/day. Citalopram may be administered in a dosage of about 20 mg/day to about 80 mg/day. Fluvoxamine may be administered in a dosage of about 50 mg/day to about 300 mg/day. The serotonin reuptake inhibitor may also be a serotonin, norepinephrine reuptake inhibitor (SNRI). Examples of SNRIs include but are not limited to duloxetine, venlafaxine, desvenlafaxine, and milnacipran. Duloxetine may be administered in a dosage of about 20 mg/day to about 60 mg/day. Venlafaxine may be administered in a dosage of about 25 mg/day to about 150 mg/day. Desvenlafaxine may be administered in a dosage of about 50 mg/day to about 100 mg/day. Milnacipran may be administered in a dosage of about 12.5 mg/day to about 100 mg/day

Where the method includes administering a neuroleptic or antipsychotic in conjunction with ondansetron, the neuroleptic or antipsychotic may be, but is not limited to, olanzapine, quetiapine, ziprasidone, haloperidol (a.k.a. aloperidol), aripiprazole, paliperidone, and risperidone. Olanzapine may be administered in a dosage of about 2.5 mg/day to about 40 mg/day. Quetiapine may be administered in a dosage of about 150 mg/day to about 750 mg/day. Ziprasidone may be administered in a dosage of about 20 mg/day to about 200 mg/day. Haloperidol (or aloperidol) may be administered in a dosage of about 2 mg/day to about 10 mg/day. Aripiprazole may be administered in a dosage of about 5 mg/day to about 30 mg/day. Risperidone may be administered in a dosage of about 0.5 mg/day to about 4 mg/day.

Each of the serotonin reuptake inhibitors, neuroleptic/antipsychotic, and ondansetron dosages may be administered orally (i.e., in swallow form). Each of the serotonin reuptake inhibitors, neuroleptic/antipsychotic, and ondansetron dosages may alternatively be administered by intracavity absorption—e.g., in the oral cavity. Administration may include, but is not limited to, absorption across the oral, sublingual, and buccal mucosas. The serotonin reuptake inhibitors, neuroleptic/antipsychotic, and ondansetron may be administered in the same dosage form, in separate dosage forms, or in various combinations.

Daily low doses of ondansetron were found to be beneficial in treating OCD hitherto shown to be too difficult to respond to SRI followed by neuroleptic augmentation of the SRI therapy. For example, in a single-blind prospective study nine (9) out of 14 adult patients with a DSM IV diagnosis of treatment-resistant OCD under stable treatment with SRI and a neuroleptic augmentation experienced significant improvement in their OCD symptoms within 12 weeks of treatment. Sixty-four percent of this small sample of treatment resistant OCD patients experienced a greater than 25% improvement in their OCD symptoms. The OCD symptoms as measured by the YBOCS scale improved by 16% at week 6 and 23% at week 12 following the ondansetron therapy.

Daily low doses of ondansetron were also found to be beneficial in treating OCD hitherto shown to be too difficult to respond to SRI therapy. For example, twenty-one patients with a DSM-IV diagnosis of treatment-resistant OCD received 12 weeks of single-blind ondansetron augmentation initiated at a dose of 0.25 mg twice daily for 2 weeks, and titrated to 0.5 mg twice daily for 10 weeks. Twelve of the 21 (57%) patients in the study experienced a treatment response in 12 weeks or less. The average reduction in YBOCS-rated symptoms of the whole group was 26.3%. The average reduction in CGI scores for the whole group was 46%. Furthermore, during the discontinuation phase (after Week 12), the YBOCS symptoms worsened by 14.6% in all patients and 38.3% in responders.

### BRIEF DESCRIPTION OF THE DRAWINGS

FIG. 1 shows the distribution of mean YBOCS scores with standard error bars over 12 weeks of treatment for the whole group (n=14).

FIG. 2 shows individual YBOCS scores at baseline, week 6, and week 12 of patients diagnosed with treatment-resistant OCD who initially failed to respond to both SRI therapy and risperidone augmentation of the SRI therapy.

FIG. 3 shows individual YBOCS scores at baseline, week 6, and week 12 of patients diagnosed with treatment-resistant OCD who initially failed to respond to both SRI therapy and either aripiprazole, quetiapine or haloperidol augmentation of the SRI therapy.

FIG. 4 shows the average Clinical Global Impressions-Improvement (CGI-I) scores at baseline, week 6, and week 12 in patients who demonstrated significant improvement in YBOCS scores.

FIG. 5 shows individual YBOCS scores at baseline, week 6, and week 12 of patients diagnosed with treatment-resistant OCD who initially failed to respond to SRI therapy (escitalopram, fluoxetine, sertraline, or venlafaxine).

FIG. 6 shows individual YBOCS scores at baseline, week 6, and week 12 of patients diagnosed with treatment-resistant OCD who initially failed to respond to SRI therapy (fluvoxamine).

FIG. 7 shows the percent decrease in mean YBOCS scores for all patients from week 2 through week 12.

FIG. 8 shows the percent decrease in mean YBOCS scores for the Responders and Non-Responders from week 2 through week 12.

FIG. 9 shows the average YBOCS scores for all patients from week 0 through week 16.

FIG. 10 shows the average YBOCS scores for Responders from week 0 through week 16.

### DETAILED DESCRIPTION OF THE INVENTION

Ondansetron is currently approved for the treatment of nausea and vomiting induced by chemotherapy (commonly described as chemotherapy-induced nausea and vomiting or CINV), and radiation (commonly described as radiation-induced nausea and vomiting or RINV) and nausea and vomiting that is post-operative (commonly described as post-operative nausea and vomiting or PONV).

Ondansetron is a highly selective 5-HT₃ receptor antagonist. Anti-emetic effects of ondansetron are mediated via antagonism of 5-hydroxytryptamine receptors located in the chemoreceptor trigger zone in the brain (central), and possibly also on vagal afferents in the upper gastrointestinal tract (peripheral). In animal studies, ondansetron did not appear to have effect on the mesenteric bed or the nerves in the heart suggesting that drug's effects are central rather than peripheral.

### I. EXAMPLES

The following example is offered to illustrate, but not to limit, the claimed invention.

### Example 1

### Ondansetron 0.75 mg Sublingual Tablet Composition

Individuals suffering from OCD may be administered the following ondansetron 0.75 mg (free base) sublingual tablet. A sublingual tablet of low dose of ondansetron may be prepared according to the formulation set forth in Table 1.

TABLE 1
Ondansetron sublingual tablet

Quantity
(mg/lozenge)
Strength

| Component | mg | % |
|---|---|---|
| Ondansetron HCl dihydrate | 0.94 | 0.45 |
| Pharmaburst ™ B2: | 133.56 | 63.60 |
| mannitol | | |
| sorbitol | | |
| crospovidone | | |
| silicon dioxide | | |
| (SPI Pharma, Wilmington, | | |
| DE) | | |
| Croscarmellose sodium | 10 | 4.76 |
| Sodium carbonate | 17 | 8.10 |
| Sodium bicarbonate | 23 | 10.95 |
| Natural and artificial | 3.0 | 1.43 |
| spearmint FONA# 913.004 | | |
| Silicon dioxide | 8.0 | 3.81 |
| Silicon dioxide colloidal | 2.0 | 0.95 |
| Sucralose | 1.50 | 0.71 |
| Sodium stearyl fumarate | 10.0 | 4.76 |
| Colorant | 1 | 0.48 |
| Total lozenge weight (mg) | 210 | 100 |

The sublingual tablets may be manufactured using a dry process, which includes screening, blending, and compression steps. The screening steps are performed to de-lump the active drug substance and the excipients. The sublingual tablets may be manufactured as follows:
1. Screen iron oxide and croscarmellose sodium through a 60-mesh screen into a polyethylene-lined container.
2. Screen silicon dioxide colloidal—Cabot, and Buffered Soda through a 30-mesh screen into a polyethylene-lined container.
3. Screen ondansetron hydrochloride dihydrate and the one-third of Pharmaburst™ B2 through a 30-mesh screen into a polyethylene-lined container.
4. Add screened material from steps 1 to 3 into a V-blender and mix the dry blend for 18 to 21 minutes.
5. Discharge the blend from the blender and pass through a 30-mesh screen into a polyethylene-lined container.
6. Screen natural and artificial spearmint flavor and silicon dioxide, NF, through a 30-mesh screen into a polyethylene lined container. Then screen sucralose and the second one-third portion of Pharmaburst™ B2 through a screen into the same polyethylene-lined container.
7. Add material from 5 and 6 into the V-blender and further mix the dry blend for 18 minutes to 21 minutes.
8. Screen the remaining Pharmaburst™ B2 and sodium stearyl fumarate through a 30-mesh screen into a polyethylene-lined container and add to the V-blender and further mix the dry blend for 14 to 17 minutes.
9. Discharge the blend into a bin or directly into the hopper.
10. Compress the lozenge using 11/32″ round tooling using a rotary tablet press.

### Example 2

### Ondansetron 0.4 mg Sublingual Tablet Composition

Individuals suffering from OCD may be administered the following ondansetron 0.4 mg sublingual tablet. A sublingual tablet of low dose of ondansetron may be prepared according to the formulation set forth in Table 2. The ondansetron 0.32 mg (free base) tablet may be manufactured according to the same process described with respect to Example 1.

TABLE 2
Ondansetron sublingual tablet

Quantity
(mg/lozenge)
Strength

| Component | mg | % |
|---|---|---|
| Ondansetron HCl dihydrate | 0.4 | 0.60 |
| Pharmaburst ™ B2: | 134 | 63.45 |
| mannitol | | |
| sorbitol | | |
| crospovidone | | |
| silicon dioxide | | |
| (SPI Pharma, Wilmington, | | |
| DE) | | |
| Croscarmellose sodium | 10 | 4.76 |
| Sodium carbonate | 17 | 8.10 |
| Sodium bicarbonate | 23 | 10.95 |
| Natural and artificial | 3.0 | 1.43 |
| spearmint FONA# 913.004 | | |
| Silicon dioxide | 8.0 | 3.81 |
| Silicon dioxide colloidal | 2.00 | 0.955 |
| Sucralose | 1.50 | 0.71 |
| Sodium stearyl fumarate | 10.0 | 4.76 |
| Colorant | 1 | 0.476 |
| Total lozenge weight (mg) | 210 | 100 |

### Example 3

### Ondansetron Peroral Tablet

Individuals suffering from OCD may be administered the following ondansetron peroral tablet. An immediate release tablet of low dose of ondansetron (0.4 mg free base) may be prepared according to the formulation set forth in Table 3.

TABLE 3
Low dose ondansetron tablet

| Component | Quantity (mg) |
|---|---|
| Ondansetron HCl dihydrate | 0.5 |
| Povidone K29/32 | 16.6 |
| Sodium Starch Glycolate (SSG) | 7.4 |
| Starch 1500 | 15.0 |
| Lactose Fast Flow | 82.0 |
| Prosolv SMCC 90 | 67 |
| Sodium bicarbonate | 160 |
| Magnesium Stearate | 1.5 |
| Total | 350 |

### Manufacturing Process

Dispensing: Screen the ondansetron HCl and excipients through screen #30. Dispense the required quantities of each ingredient.

Blending:
1. Transfer the ondansetron hydrochloride and Povidone K 29/32 to a V-Shell blender and blend for 2 minutes.
2. Add SSG and Starch 1500 to Step 1 and blend for another 2 minutes.
3. Add Lactose Fast Flow and Prosolv SMCC 90 to Step 2 and blend for another 10 minutes.
4. Mix an equal amount of the blend from Step 3 with magnesium stearate or sodium stearyl fumarate and transfer the mixture back to the V-Shell blender via screen #30. Blend for 3 minutes.

Compression: Compress the final blend from Step 4 on a rotary press to a target tablet weight of 350 mg.

### Example 4

### Ondansetron Oral Solution

The ondansetron oral solution included ondansetron hydrochloride dihydrate (6.25% w/v), sodium benzoate (0.2% w/v) as a preservative, sodium citrate (0.015%) as taste masking agent and lemon lime flavor (0.91% w/v) as a flavoring agent in water.

### Example 5

### Ondansetron Augmentation Therapy (SRI and Antipsychotic)

Between March 2008 and November 2008, fourteen adults aged 18 to 55 were enrolled in a study at the Institute for Neuroscience, Florence, Italy. These adults had a Yale-Brown Obsessive-Compulsive Scale (Y-BOCS) (see Goodman, W. K. et al., ARCH GEN PSYCHIATRY 1989; 46:1006-1011) score of ≧20 after ≧12 weeks of treatment with an established effective dose of an SRI or clomipramine and after ≧10 weeks of augmentation treatment with antipsychotics (risperidone at least 2 mg/day; quetiapine at least 150 mg/day; olanzapine at least 5 mg/day; haloperidol titrated at least 10 mg/day; or aripiprazole at least 10 mg/day). All diagnoses were established utilizing a structured interview, the Structured Clinical Interview for DSM IV Axis I Disorders (SCID I). (See Spitzer, R. L. et al. ARCH GEN PSYCHIATRY 49(8):624-649) Potential subjects with a history of alcohol or substance abuse, current severe depressive symptoms, bipolar disorder, panic disorder, schizophrenia, other psychiatric conditions, heart disease, arrhythmia, liver problems (including cirrhosis), seizures, glaucoma or serious medical disease were excluded. Patients with hoarding as their only OCD symptom and women of childbearing potential not using a medically acceptable contraceptive method were also excluded.

After being fully informed about the study and signing an informed consent approved by the Internal Institutional Review Board, subjects were administered an ondansetron hydrochloride (oral solution) dosage of 0.25 mg twice a day for 6 weeks, followed by 0.5 mg twice a day for 6 weeks for a total observation period of 12 weeks.

Subjects were seen at a screening followed by a baseline visit after two weeks, at which times inclusion/exclusion criteria, OCD symptoms, comorbid symptoms and vital signs were assessed. The Y-BOCS, Montgomery-Asberg Depression Rating Scale (MADRS) (see Montgomery, S. A., BR J PSYCHIATRY 1979; 134:382-389) was administered at screening and baseline, and these assessments were repeated at every follow-up visit at 2-week intervals, along with the Clinical Global Impressions-Improvement (CGI-I) rating (see Guy, W. ECDEU Assessment Manual for Psychopharmacology. US Dept Health, Education, and Welfare publication (ADM) 76-338. Rockville, Md.: National Institute of Mental Health; 1976:218-222) and an unpublished Drug Effect scale. The Drug Effect scale asked subjects to rate “How anxious are you now?” on a scale of zero (not at all) to 10 (“the most anxious I have ever been”). Similar rating questions have been “Do you experience appearance or increment in frequency of headaches?”, “Do you experience appearance or increment in frequency of headaches fatigue?” and “Do you experience appearance or increment in frequency of headaches constipation” and so on regarding other common ondansetron side effects (diarrhea, urinary retention, itching, and dizziness). Furthermore, each evening during the first week of study medication, a study psychiatrist called the subject to ask for major or unexpected collateral effects (as chest pain, an unexplained skin rash, or tremors.). Criteria suggested by Mittman and colleagues was used to classify subjects as having mild depression (MADRS score 9-17) or at least moderate depression (MADRS scores ≧18). (See Mittmann, N. et al., Am J PSYCHIATRY 1997; 154:320-321) Subjects with MADRS scores ≧18 were excluded. Ratings were administered by an investigator who was blind to the treatment condition of the patients.

Given the small sample size, mean changes from baseline in rating scales were tested with a non-parametric method based on ranks (Wilcoxon signed rank test), using a two-tailed significance level of p≦0.05. Relationships among rated and demographical variables were tested with Spearman's rank correlation tests, using a two-tailed significance level of p≦0.05. Age, baseline OCD severity, and depressive symptoms were controlled for by stratifying through the Mantel-Haenszel χ2 test. Body mass index (BMI) was adjusted given the differences of ondansetron bioavailability according to BMI.

### Results

A total of 14 Caucasian subjects (6 women and 8 men) with a mean age of 42.2 (±10.8) years (range: 19 to 55 years), with a mean duration of illness of 14.1 (+4.07) years (range: 3 to 26 years) were enrolled. An additional 20 individuals screened were not enrolled: 6 patients, after hearing details of the study, were not interested and 14 patients were not eligible (exclusionary comorbid condition, 9; inadequate anti-OCD medication dose/duration, 3; insufficient OCD severity, 2).

Subjects' clinical characteristics and results are presented in Tables 4, 5, and 6.

TABLE 4
Baseline Clinical Characteristics and Subsequent Clinical Measures in
OCD Subjects Receiving Ondansetron responder and non responder
(n = 14)

| | Responder (n = 9) | Non responder (n = 5) |
|---|---|---|
| Gender | 3 F; 6 M | 3 F; 2 M |
| Age (mean, SD) | 41.8 (11.1) | 42.8 (11.4) |
| Duration of OCD (mean, SD) | 14.8 (5.3) | 12.8 (3.7) |
| BMI (mean, SD) | 23 (5.8) | 21.8 (1.4) |
| Baseline score (mean, SD) | | |
| YBOCS | 29.5 (6.4) | 24 4 (4.5) |
| CGI | 4.8 (0.7) | 5.2 (1.3) |
| MADRS | 7.0 (2.3) | 7.8 (3.8) |
| End of 6^{th} week (mean, SD) | | |
| YBOCS | 22.6 (4.2) | 22.8 (3.8) |
| CGI | 3.4 (1.6) | 4.6 (1.5) |
| MADRS | 6.7 (2.4) | 8.0 (4.0) |
| End of 12^{th} week (mean, SD) | | |
| YBOCS | 20.8 (4.1) | 21.4 (6.1) |
| CGI | 2.3 (1.2) | 4.8 (1.3) |
| MADRS | 6.8 (2.0) | 7.6 (3.8) |

TABLE 5
YBOCS Scores for the 14 Patients

| | | | | Percent | Percent |
|---|---|---|---|---|---|
| Patient | | | | Reduction at | Reduction at |
| No. | Baseline | 6 weeks | 12 weeks | 6 weeks | 12 weeks |
| 1 | 19 | 17 | 15 | 10.52 | 21.05 |
| 2 | 24 | 24 | 20 | .00 | 16.60 |
| 3 | 31 | 25 | 22 | 19.32 | 29.03 |
| 4 | 26 | 25 | 25 | 3.84 | 3.84 |
| 5 | 35 | 25 | 25 | 28.57 | 28.57 |
| 6 | 35 | 28 | 25 | 20.00 | 28.57 |
| 7 | 20 | 17 | 15 | 15.00 | 25.00 |
| 8 | 20 | 16 | 14 | 20.00 | 30.00 |
| 9 | 31 | 27 | 30 | 12.90 | 3.22 |
| 10 | 33 | 26 | 23 | 21.20 | 27.27 |
| 11 | 35 | 24 | 22 | 31.42 | 37.14 |
| 12 | 33 | 24 | 24 | 27.27 | 27.27 |
| 13 | 24 | 19 | 18 | 20.80 | 25.00 |
| 14 | 22 | 21 | 17 | 4.54 | 22.72 |

TABLE 6
SRI + Neuroleptic Treatment Regimens for the 14 Patients

| | Selective | | | |
|---|---|---|---|---|
| | Serotonin | | | |
| Patient | Reuptake | Neuroleptic/ | | Non- |
| No. | Inhibitor | Antipsychotic | Responder | Responder |
| 1 | fluvoxamine | aloperidol 10 mg/day | | X |
| | 250 mg/day | | | |
| 2 | paroxetine | aripiprazole 15 mg/day | | X |
| | 40 mg/day | | | |
| 3 | fluvoxamine | aloperidol 10 mg/day | X | |
| | 250 mg/day | | | |
| 4 | fluvoxamine | quetiapine 200 mg/day | | X |
| | 250 mg/day | | | |
| 5 | citalopram | risperidone 2 mg/day | X | |
| | 40 mg/day | | | |
| 6 | fluvoxamine | risperidone 2 mg/day | X | |
| | 250 mg/day | | | |
| 7 | paroxetine | risperidone 2 mg/day | X | |
| | 40 mg/day | | | |
| 8 | paroxetine | aripiprazole 15 mg/day | X | |
| | 40 mg/day | | | |
| 9 | citalopram | quetiapine 200 mg/day | | X |
| | 40 mg/day | | | |
| 10 | fluvoxamine | quetiapine 200 mg/day | X | |
| | 250 mg/day | | | |
| 11 | citalopram | risperidone 2 mg/day | X | |
| | 60 mg/day | | | |
| 12 | citalopram | risperidone 2 mg/day | X | |
| | 60 mg/day | | | |
| 13 | fluoxetine | risperidone 3 mg/day | X | |
| | 40 mg/day | | | |
| 14 | paroxetine | risperidone 2 mg/day | | X |
| | 40 mg/day | | | |

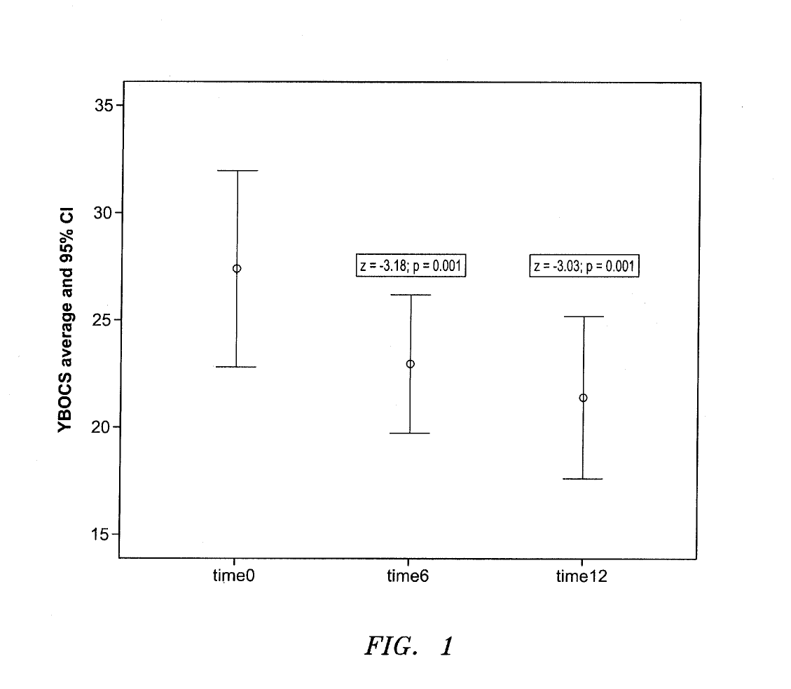

As seen in Table 5, at the end of the 6^{th} week, three of the fourteen patients (3/14; 21.4%) (Patients 5, 11, and 12) of the OCD patients met the YBOCS criteria for “treatment response” (reduction >25%). (See Pallanti, S. et al., INT J NEUROPSYCHOPHARMACOL. 2002 June; 5(2):181-91) The average YBOCS score change of the whole group at 6 weeks was a 16% decrease (range: 0% increase to 31% decrease), which was statistically significant (Sum of Rank=91; z=−3.18; p=0.001) (FIG. 1). None of the fourteen subjects experienced significant side effects or worsening of OC symptoms.

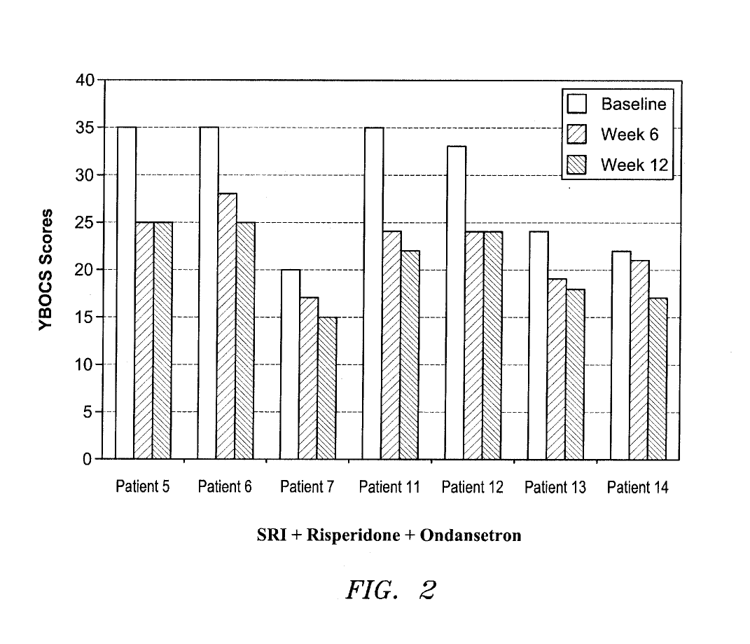

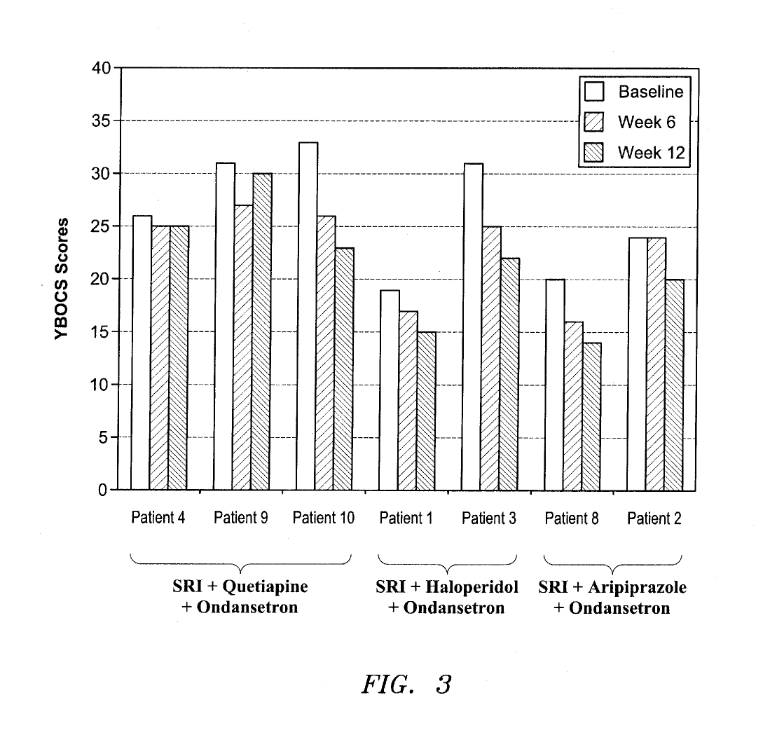

The YBOCS score continued to improve from the 6^{th} until the 12^{th} week. At the 12^{th} week, nine of the fourteen patients (9/14; 64.2%) reached the YBOCS criteria for “treatment response” (a YBOCS score decrease of ≧25%) and a CGI-I score decrease to 1 or 2 (seven patients were either “very much improved” or “much improved”). FIG. 2 shows the YBOCS scores at baseline, week 6, and week 12 for patients treated with an SRI, risperidone, and ondansetron. Notably, six of the seven patients (86%) on risperidone were responders to ondansetron. One subject (Patient 11) reached a YBOCS score decrease of 35% (in treatment with citalopram 60 mg/day and risperidone 2 mg/day). FIG. 3 shows the YBOCS scores at baseline, week 6, and week 12 for patients treated with an SRI, ondansetron, and one of quetiapine, aripiprazole, and haloperidol. Of the 14 patients treated, three female and six male patients were treatment responders. Baseline SRI and neuroleptic treatments for the 9 adjunctive ondansetron responder patients are listed in Table 6.

The average YBOCS change score of the whole group at the end of 12 weeks was a 23.2% decrease (range: 3.2% increase to 37.4% decrease of YBOCS scores). The improving YBOCS score trend at the 12^{th} week was still significant compared to baseline (Sum of Rank=105; z=−3.03; p=0.001) (FIG. 1). One subject experienced slight worsening of OC symptoms from week 11 until week 12 in association with a gastrointestinal infection.

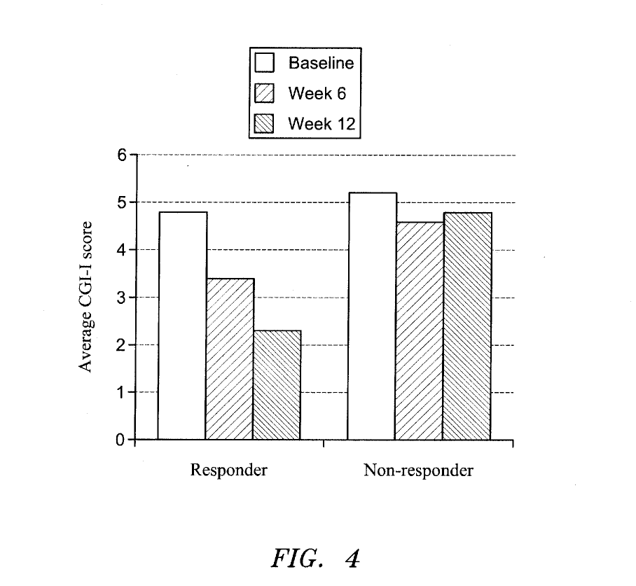

The Clinical Global Impressions-Improvement (CGI-I) scores improved concurrent with improvement in the YBOCS scores. The improvement persisted beyond week six, and continued to improve through week 12, the last assessment point of the study as shown in FIG. 4.

Spearman's non parametric test did not show any significant correlations between percent change in Y-BOCS scores and other demographical or clinical variables. Mann-Whitney U comparisons showed no significant differences between responder and non responder in demographical variables means such as age (U=22.00; z=−0.06; Exact p=1.0), BMI (U=19.00; z=−1.54; Exact p=0.147), duration of illness (U=16.5; z=−0.81; Exact p=0.699) and clinical variable such as baseline YBOCS (U=11.00; z=−1.54; Exact p=0.122), CGI (U=16.00; z=−0.91; Exact p=0.438) and MADRS values (U=19; z=−0.47; Exact p=0.699). Mean MADRS scores did not change significantly from baseline to the end of week 1.

Furthermore, Mantel-Haenszel χ2 test showed no effect on the outcome stratified by gender (Mantel Haenszel=0.103; df=1; p=0.74), baseline severity (Mantel Haenszel=1.156; df=1; p=0.28), duration of illness (Mantel Haenszel=0.007; df=1; p=0.93), age (Mantel Haenszel=0.00; df=1; p=1.00), and BMI (Mantel Haenszel=0.506; df=1; p=0.45). Therefore, there did not appear to be evidence of a possible role of demographical and clinical variables in the ondansetron response.

Side effects throughout the study were mild to moderate and included decreased appetite (two patients for the first 2 weeks of treatment), and headache (two patients for the first 2 weeks of treatment). One patient reported a mild increase in anxiety levels, which lasted only one night. No change in mean pulse rate or mean blood pressure was observed. None of the subjects experienced significant or lasting side effects (such as constipation), likely due to the very low and slow titration of the medication. The daily dose of 1 mg/day divided in twice daily administration, approximately 5% of the minimum dose recommended for emesis and nausea treatment was reached after 6 weeks and maintained until the 12^{th} week.

### Discussion

The study results suggest ondansetron as an effective adjunct treatment in OCD patients resistant to SRI plus antipsychotic therapy. YBOCS OCD symptoms were significantly reduced at 12 weeks as a result of adding ondansetron to the existing SRI and neuroleptic/antipsychotic treatment therapy. Sixty-four percent of this sample of treatment-resistant-OCD patients experienced a greater than 25% additional response of OCD severity, and the sample as a whole experienced a significant symptom reduction on YBOCS severity score of 16% at week 6 and 23% at week 12 of ondansetron augmentation treatment (FIG. 1).

This prospective single blind pilot trial demonstrates efficacy of ondansetron augmentation in treatment resistant OCD patients treated with SRI plus neuroleptic/antipsychotic treatment. Although there is a possibility that the symptom improvement was due to a late onset of the antipsychotic augmentation, the improvement was seen in the second half of the trial. This makes it less likely that the observed improvements were attributable to the stable dose of antipsychotic treatment. Moreover, two responder patients, after discontinuation of the ondansetron, experienced worsening and asked to go back on ondansetron.

### Example 6

### Ondansetron Augmentation Therapy (SRI)

In the previous study, it was shown that ondansetron was useful as an augmentation to antipsychotic+SRI combination treatment. In this study, ondansetron as an augmentation therapy to SRI monotherapy for treatment-resistant OCD was investigated.

Twenty-one patients with a DSM-IV diagnosis of treatment-resistant OCD, under stable treatment with SRIs approved by the FDA for OCD therapy, received 12 weeks of single-blind ondansetron augmentation initiated at a dose of 0.25 mg twice daily for 2 weeks, and titrated to 0.5 mg twice daily for 10 weeks. Patients were rated every two weeks using the Yale Brown Obsessive Compulsive Scale (YBOCS) and Clinical Global Impressions Scale (CGI). Treatment-resistant patients were defined as having completed an adequate trial of SRIs at a moderate to high dose for at least 12 weeks and still having a YBOCS severity of >24, and CGI-Severity of ≧4. Treatment response was defined as an additional 25% reduction in YBOCS score from YBOCS score at the initiation of ondansetron augmentation, an end of treatment (EOT) period YBOCS of ≦24, and CGI-Improvement (CGI-I) of ≦2. Additionally, ondansetron was discontinued after 12 weeks and patients were followed up for another 4 weeks for relapse in YBOCs symptoms.

### Results

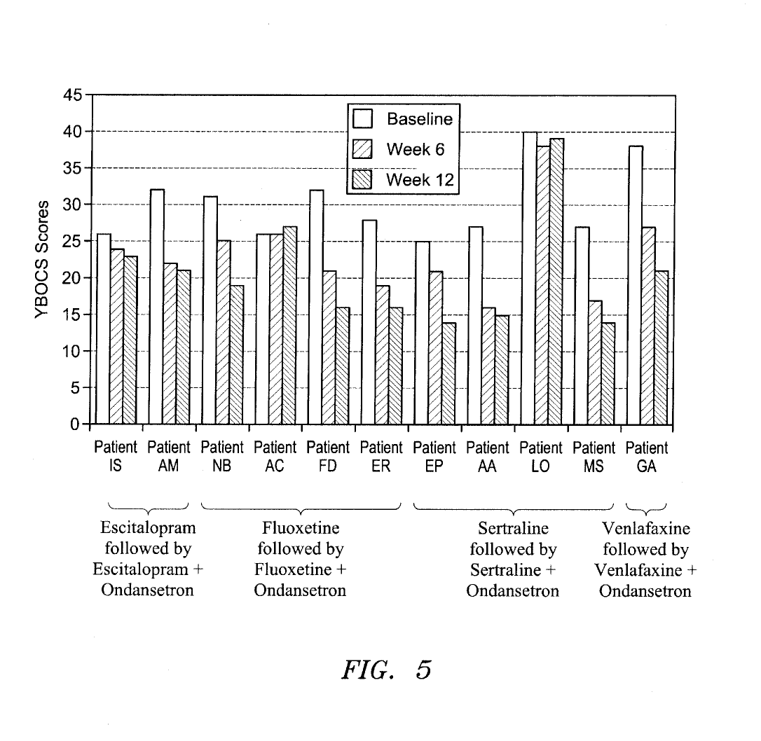

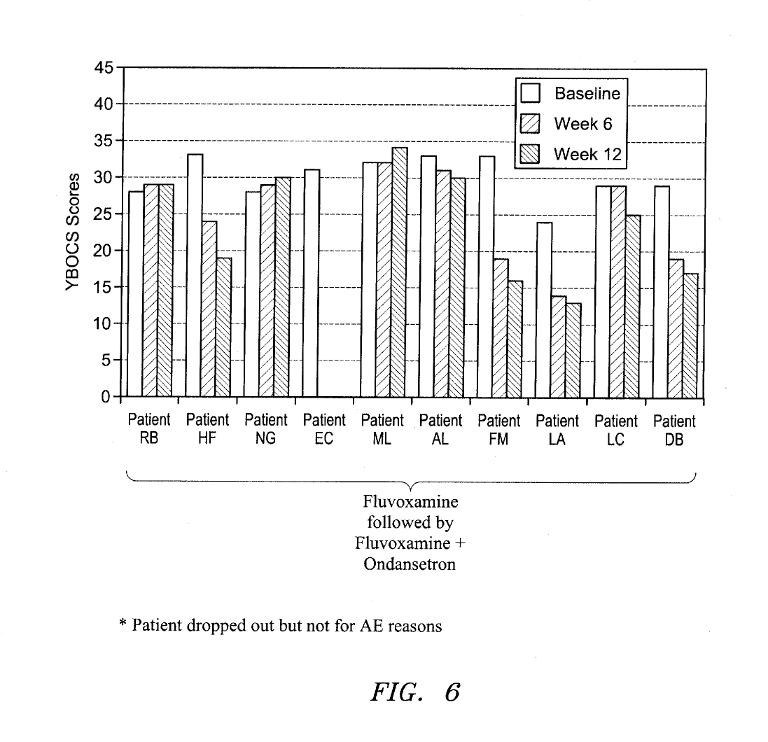

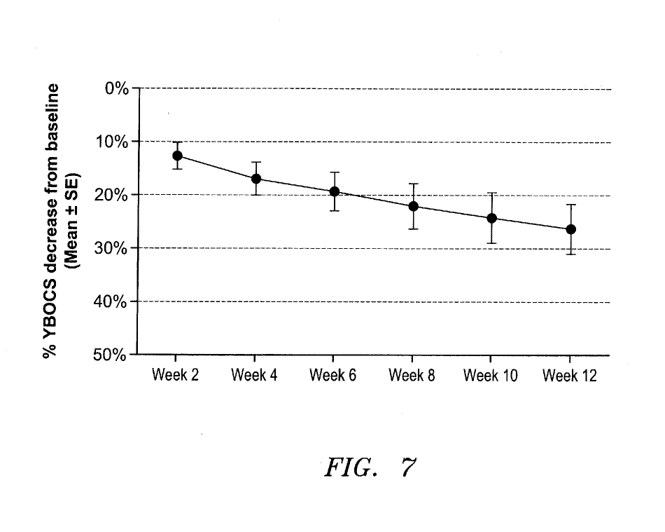

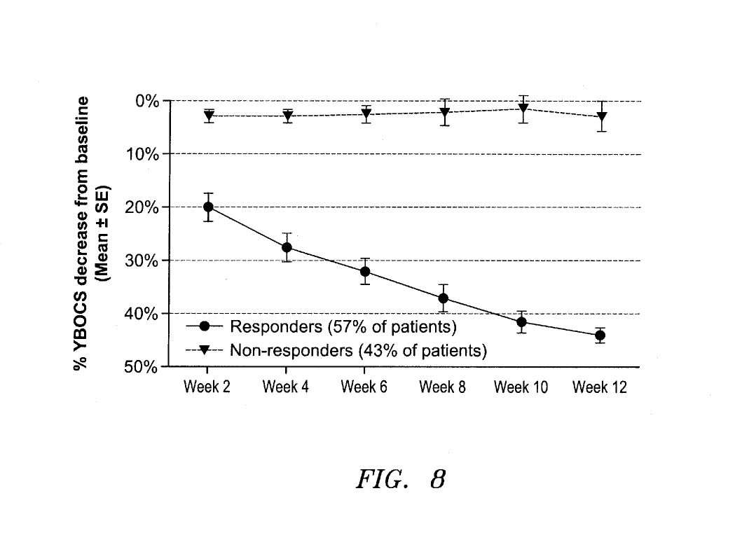

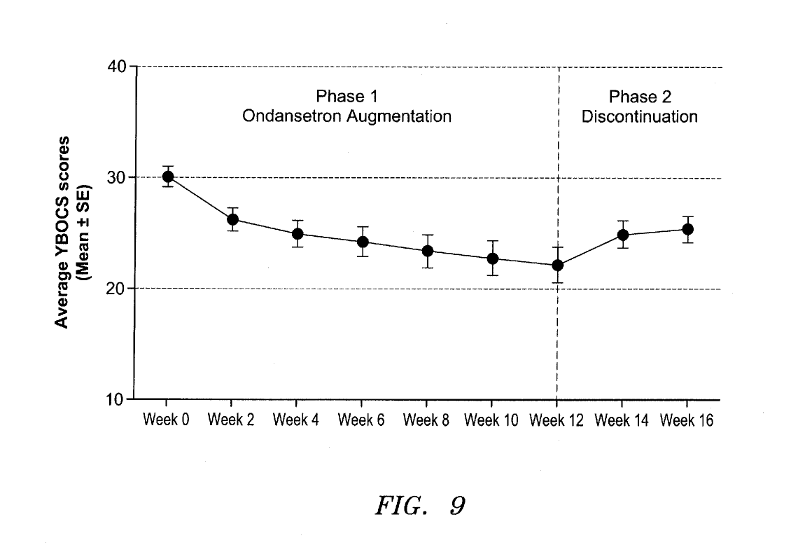

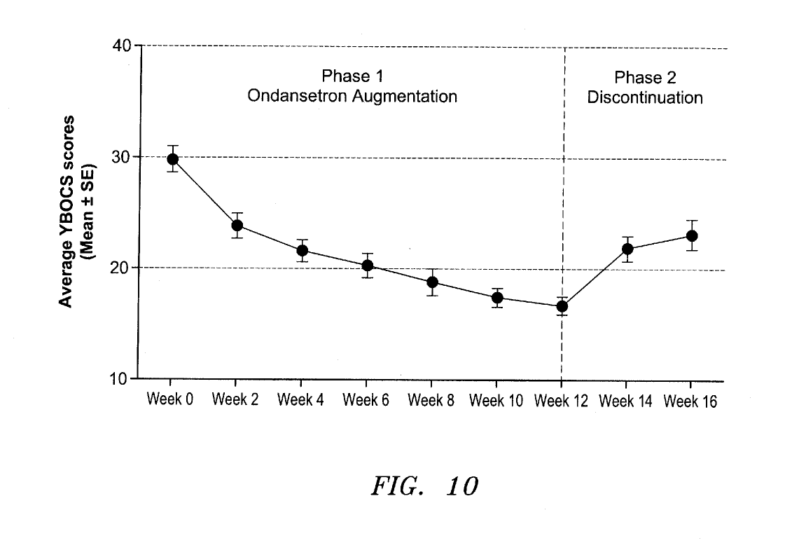

Subjects' clinical characteristics and results are presented in Tables 7-10. Treatment response was defined as a 25% reduction in YBOCS score from initiation of ondansetron augmentation, with an EOT period YBOCS of <24 and CGI-I of 1-2. As seen in Table 8, eight (38%) patients experienced response within the first 6 weeks based on 25% improvement in YBOCS, EOT YBOCS <24, and CGI-I=1-2. The YBOCS scores continued to improve from the 6^{th} until the 12^{th} week. FIGS. 5 and 6 show the YBOCS scores at baseline, week 6, and week 12 for patients treated with the specified SRI and ondansetron. Twelve of the 21 (57%) patients in the study experienced a treatment response in 12 weeks or less. (See Table 9.) The average reduction in YBOCS-rated symptoms of the whole group was 26.3%. (See FIG. 7.) The average reduction in CGI scores for the whole group was 46%. As seen in FIG. 8, responders had a 44% reduction in YBOCS and 77% reduction in CGI-I from baseline after 12 weeks. Non-responders showed marginal improvement in YBOCS scores after 12 weeks. There was a 2.9% difference, between YBOCS score at baseline and after 12 weeks. During the discontinuation phase (after Week 12), the YBOCS symptoms worsened by 14.6% in all patients (FIGS. 9) and 38.3% in responders (FIG. 10). Treatment was well tolerated.

TABLE 7
Baseline Clinical Characteristics and Subsequent Clinical Measures in
OCD Subjects Receiving Ondansetron - Responder and Non-Responder

| | | Non |
|---|---|---|
| | Responder (n = 12) | responder (n = 9) |
| Gender | 6 F; 6 M | 5 F; 4 M |
| Age (mean, SD) | 37.1 (8.4) | 35.6 (13.1) |
| Duration of OCD (mean, SD) | 10.08 (4.6) | 10.6 (9.3) |
| Baseline score (mean, SD) | | |
| YBOCS | 29.92 (4.0) | 30.33 (4.4) |
| CGI | 5.08 (0.9) | 5.3 (0.5) |
| End of 6^{th} week (mean, SD) | | |
| YBOCS | 20.33 (3.8) | 29.56 (4.0) |
| CGI | 1.92 (0.7) | 4.63 (0.5) |
| End of 12^{th} week (mean, SD) | | |
| YBOCS | 16.75 (2.7) | 29.44 (4.8) |
| CGI | 1.17 (0.4) | 4.88 (0.4) |

TABLE 8
YBOCS and CGI-I Scores for the 21 Patients

| | Baseline | | 6 Weeks | | | 12 Weeks | | |
|---|---|---|---|---|---|---|---|---|
| | YBOCS | CGI-I | YBOCS | YBOCS % | CGI-I | YBOCS | YBOCS % | CGI-I |
| Patient | Score | Score | Score | Reduction | Score | Score | Reduction | Score |
| AA | 27 | 5 | 16 | 40.74 | 1 | 15 | 44.44 | 1 |
| AC | 26 | 5 | 26 | 0.00 | 4 | 27 | −3.85 | 5 |
| AL | 33 | 5 | 31 | 6.06 | 5 | 30 | 9.09 | 5 |
| AM | 32 | 5 | 22 | 31.25 | 2 | 21 | 34.38 | 1 |
| DB | 29 | 5 | 19 | 34.48 | 3 | 17 | 41.38 | 1 |
| EC* | 31 | 6 | 28 | 9.68 | 5 | 28 | 9.68 | 5 |
| EP | 25 | 5 | 21 | 16.00 | 2 | 14 | 44.00 | 1 |
| ER | 28 | 5 | 19 | 32.14 | 3 | 16 | 42.86 | 1 |
| FD | 32 | 5 | 21 | 34.38 | 1 | 16 | 50.00 | 1 |
| FM | 33 | 5 | 19 | 42.42 | 1 | 16 | 51.52 | 1 |
| GA | 38 | 6 | 27 | 28.95 | 2 | 21 | 44.74 | 1 |
| HF | 33 | 5 | 24 | 27.27 | 2 | 19 | 42.42 | 1 |
| IS | 26 | 5 | 24 | 7.69 | 4 | 23 | 11.54 | 4 |
| LA | 24 | 5 | 14 | 41.67 | 2 | 13 | 45.83 | 2 |
| LC | 29 | 5 | 29 | 0.00 | 4 | 25 | 13.79 | 5 |
| LO | 40 | 6 | 38 | 5.00 | 5 | 39 | 2.50 | 5 |
| ML | 32 | 5 | 32 | 0.00 | 5 | 34 | −6.25 | 5 |
| MS | 27 | 4 | 17 | 37.04 | 2 | 14 | 48.15 | 1 |
| NB | 31 | 6 | 25 | 19.35 | 2 | 19 | 38.71 | 2 |
| NG | 28 | 5 | 29 | −3.57 | 5 | 30 | −7.14 | 5 |
| RB | 28 | 6 | 29 | −3.57 | 5 | 29 | −3.57 | 5 |

*This subject dropped out after 1 week of treatment for reasons not known. The YBOCS score at the end of week 1 (28) was carried forward for week 6 and week 12.

TABLE 9
SRI Treatment Regimens for the 21 Patients

| | Selective Serotonin | | Non- |
|---|---|---|---|
| Patient | Reuptake Inhibitor | Responder* | Responder |
| AA | sertraline 250 mg | X | |
| LA | fluvoxamine 250 mg | X | |
| GA | venlafaxine 250 mg | X | |
| NB | fluoxetine 60 mg | X | |
| RB | fluvoxamine 250 mg | | X |
| DB | fluvoxamine 250 mg | X | |
| EC | fluvoxamine 200 mg | | X |
| AC | fluoxetine 60 mg | | X |
| LC | fluvoxamine 250 mg | | X |
| FD | fluoxetine 60 mg | X | |
| HF | fluvoxamine 250 mg | X | |
| NG | fluvoxamine 250 mg | | X |
| ML | fluvoxamine 200 mg | | X |
| AL | fluvoxamine 200 mg | | X |
| FM | fluvoxamine 200 mg | X | |
| AM | escitalopram 60 mg | X | |
| LO | sertraline 250 mg | | X |
| EP | sertraline 100 mg | X | |
| ER | fluoxetine 60 mg | X | |
| IS | escitalopram 40 mg | | X |
| MS | sertraline 250 mg | X | |

*Treatment response was defined as a 25% reduction in YBOCS score from initiation of ondansetron augmentation, with an EOT period YBOCS of <24 and CGI-I of 1-2.

TABLE 10
Percent Decrease in YBOCS Scores for All Patients,
Responders, and Non-Responders

| | All Patients | | Responders | | Non-Responders | |
|---|---|---|---|---|---|---|
| Week | % Decrease | SE | % Decrease | SE | % Decrease | SE |
| 2 | 12.66 | 2.47 | 20.06 | 2.62 | 2.93 | 1.23 |
| 4 | 16.93 | 3.16 | 27.58 | 2.7 | 2.93 | 1.2 |
| 6 | 19.3 | 3.62 | 32.03 | 2.41 | 2.56 | 1.62 |
| 8 | 21.99 | 4.28 | 37.05 | 2.51 | 2.2 | 2.49 |
| 10 | 24.21 | 4.73 | 41.5 | 2.04 | 1.47 | 2.73 |
| 12 | 26.27 | 4.76 | 44.01 | 1.36 | 2.93 | 2.76 |

TABLE 11
Average YBOCS Scores for All Patients

| | Avg | |
|---|---|---|
| Week | YBOCS Scores | SE |
| 0 | 30.1 | 0.89 |
| 2 | 26.29 | 1.01 |
| 4 | 25 | 1.16 |
| 6 | 24.29 | 1.31 |
| 8 | 23.48 | 1.49 |
| 10 | 22.81 | 1.58 |
| 12 | 22.19 | 1.61 |
| 14 | 24.95 | 1.21 |
| 16 | 25.43 | 1.18 |

These efficacy, safety, and relapse data suggest that patients who do not adequately respond to SRI treatment may benefit from augmentation with a low dose of ondansetron and possibly avoid the use of anti-psychotic augmentation.

### Example 7

### B.I.D. Dosing

Ondansetron may be administered according to a twice-a-day (b.i.d.) dosage regimen. For example, ondansetron may be administered in two doses separated by at least about 9 hours, alternatively by at least about 10 hours, alternatively by at least about 10.5 hours, alternatively by at least about 9 hours to about 12 hours.

All publications, patent applications, and patents cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating obsessive compulsive disorder, comprising the steps of:
administering a serotonin reuptake inhibitor and ondansetron or a pharmaceutically acceptable salt thereof to a patient suffering from obsessive compulsive disorder, wherein the ondansetron or pharmaceutically acceptable salt thereof is administered as a pharmaceutically effective dose up to about 1.5 mg per day (free-base equivalent); and
repeating the step of administering the serotonin reuptake inhibitor and the ondansetron or a pharmaceutically acceptable salt thereof for more than seven days.

2. The method of claim 1, wherein the patient suffers from treatment-resistant obsessive compulsive disorder.

3. The method of claim 1, wherein the ondansetron or pharmaceutically acceptable salt thereof is administered in a single daily dose.

4. The method of claim 1, wherein the ondansetron or pharmaceutically acceptable salt thereof is administered twice a day.

5. The method of claim 1, wherein the ondansetron or pharmaceutically acceptable salt thereof is administered three times a day.

6. The method of claim 1, wherein the serotonin reuptake inhibitor is clomipramine.

7. The method of claim 1, wherein the serotonin reuptake inhibitor is selected from the group consisting of paroxetine, sertraline, fluoxetine, escitalopram, citalopram, and fluvoxamine.

8. The method of claim 1, wherein the ondansetron or pharmaceutically acceptable salt thereof is administered as a pharmaceutically effective dose of about 0.25 mg/day to about 1.5 mg/day.

9. The method of claim 1, wherein the ondansetron or pharmaceutically acceptable salt thereof is administered as a pharmaceutically effective dose of about 0.5 mg/day to about 1.5 mg/day.

10. The method of claim 1, wherein the ondansetron or pharmaceutically acceptable salt thereof is administered as a pharmaceutically effective dose of about 1 mg/day.

11. The method of claim 10, wherein the about 1 mg/day dose is administered as a dosage of 0.5 mg twice daily.

12. The method of claim 1, wherein the ondansetron or pharmaceutically acceptable salt thereof is administered as a pharmaceutically effective dose of about 1.5 mg/day.

13. The method of claim 12, wherein the ondansetron or pharmaceutically acceptable salt thereof is administered twice a day.

14. The method of claim 1, wherein the ondansetron or pharmaceutically acceptable salt thereof is administered orally.
